SEQUENCE LISTING (31 sequences)

SEQ ID NO: 1 - protein, 9 aa, Homo sapiens
FLSFHISNL

SEQ ID NO: 2 - protein, 21 aa, Homo sapiens
NSSLEDPSTDYYQELQRDISE

SEQ ID NO: 3 - protein, 9 aa, Homo sapiens
NLTISDVSV

SEQ ID NO: 4 - protein, 20 aa, Homo sapiens
APDTRPAPGSTAPPAHGVTS

SEQ ID NO: 5 - protein, 20 aa, Homo sapiens
APATEPASGSAATWGQDVTS

SEQ ID NO: 6 - protein, 20 aa, Homo sapiens
VPVTRPALGSTTPPAHDVTS

SEQ ID NO: 7 - protein, 20 aa, Homo sapiens
APDNKPAPGSTAPPAHGVTS

SEQ ID NO: 8 - protein, 20 aa, Homo sapiens
APDNRPALGSTAPPVHNVTS

SEQ ID NO: 9 - DNA, 1774 bp, Homo sapiens
ggatccgctccacctctcaagcagccagcgcctgcctgaatctgttctgccccctccccacccatttcaccaccaccatgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacaccgtccttcagaggccccaccaatttctcggacacttctcagtgtgtggaagctcatgtgggcccctgaggctcatgcctgggaagtgttgtggtgggggctcccaggaggactggcccagagagccctgagatagcggggatcc

SEQ ID NO: 10 - protein, 475 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 11 - DNA, 1164 bp, Homo sapiens
gctagcgccaccatgtctagaacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggctagcgcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtctagatagctcgag

SEQ ID NO: 12 - protein, 381 aa, Homo sapiens
MSRTPGTQSPFFLLLLLTVLTVVTGSGHASSTPGGEKETSATQRSSVPSSTEKNAVSMTSSVLSSHSPGSGSSTTQGQDVTLASASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANLSR

SEQ ID NO: 13 - DNA, 1404 bp, Homo sapiens
gctagcgccaccatgtctagaacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccgctagcccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtctagatagctcgag

SEQ ID NO: 14 - protein, 461 aa, Homo sapiens
MSRTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPASPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANLSR

SEQ ID NO: 15 - DNA, 45 bp, Homo sapiens
gcaggctagcgccaccatgtctagaacaccgggcacccagtctcc

SEQ ID NO: 16 - DNA, 37 bp, Homo sapiens
gacgctcgagctatctagacaagttggcagaagtggc

SEQ ID NO: 17 - DNA, 44 bp, Homo sapiens
ggacaggatgtcactctggctagcgcctcaggctctgcatcagg

SEQ ID NO: 18 - DNA, 44 bp, Homo sapiens
cctgatgcagagcctgaggcgctagccagagtgacatcctgtcc

SEQ ID NO: 19 - DNA, 30 bp, Homo sapiens
tacggctagcgggggcggtggagcccgggg

SEQ ID NO: 20 - DNA, 31 bp, Homo sapiens
tacggctagcccagcccatggtgtcacctcg

SEQ ID NO: 21 - DNA, 20 bp, Homo sapiens
atgacaccgggcacccagtc

SEQ ID NO: 22 - DNA, 18 bp, Homo sapiens
gaccagcagcgtactctc

SEQ ID NO: 23 - DNA, 20 bp, Homo sapiens
ccagccagcaagagcactcc

SEQ ID NO: 24 - DNA, 20 bp, Homo sapiens
cctctctggaagatcccagc

SEQ ID NO: 25 - DNA, 19 bp, Homo sapiens
ggttgcgctggccattgtc

SEQ ID NO: 26 - DNA, 20 bp, Homo sapiens
gcagaagtggctgccactgc

SEQ ID NO: 27 - DNA, 21 bp, Homo sapiens
gcactgacagacagccaaggc

SEQ ID NO: 28 - DNA, 21 bp, Homo sapiens
ccttctcggaaggccagagtc

SEQ ID NO: 29 - DNA, 21 bp, Homo sapiens
gtaccgtgctatggtgagtgc

SEQ ID NO: 30 - DNA, 21 bp, Homo sapiens
caccagagtagaagctgagcc

SEQ ID NO: 31 - DNA, 19 bp, Homo sapiens
ggagagtacgctgctggtc

(19) United States
(12) Patent Application Publication
(10) Pub. No.: US 2006/0062798 A1
(43) Pub. Date: Mar. 23, 2006

(54) Vaccines

(76) Inventors: Michael Neil Burden, Hertfordshire (GB); Paul Hamblin, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/515,872

(22) PCT Filed: May 23, 2003
(86) PCT No.: PCT/EP03/05595

§ 371 Date: Aug. 2, 2005

(30) Foreign Application Priority Data
May 24, 2002 (GB) 0212036.8

(51) Int. Cl. A61K 48/00; A61K 39/00; C07H 21/04; C07K 14/705; C07K 14/82; A61K 33/24

(52) U.S. Cl. 424/185.1; 514/44; 435/69.3; 435/320.1; 435/325; 530/350; 536/23.5; 424/649

(57) ABSTRACT

The present invention provides nucleic acid constructs useful in nucleic acid vaccination for the treatment and prophylaxis of Muc-1 expressing tumours. The constructs are devoid of Muc-1 perfect repeats.

The present invention relates to the novel nucleic acid constructs, useful in nucleic acid vaccination protocols for the treatment and prophylaxis of MUC-1 expressing tumours. In particular, the nucleic acid is DNA and the DNA constructs comprise a gene encoding a MUC-1 derivative devoid of any perfect repeats. The invention further provides pharmaceutical compositions comprising said constructs, particularly pharmaceutical compositions adapted for particle mediated delivery, methods for producing them, and their use in medicine. Novel proteins encoded by the nucleic acid and pharmaceutical compositions containing them are also provided.

### BACKGROUND TO THE INVENTION

The epithelial cell mucin MUC-1 (also known as episialin or polymorphic epithelial mucin, PEM) is a large molecular-weight glycoprotein expressed on many epithelial cells. The protein consists of a cytoplasmic tail, a transmembrane domain and a variable number of tandem repeats of a 20 amino acid motif (herein termed the VNTR monomer, it may also be known as the VNTR epitope, or the VNTR repeat) containing a high proportion of proline, serine and threonine residues. The number of repeats is variable due to genetic polymorphism at the MUC-1 locus, and most frequently lies within the range 30-100 (Swallow et al, 1987, Nature 328:82-84). In normal ductal epithelia, the MUC-1 protein is found only on the apical surface of the cell, exposed to the duct lumen (Graham et al, 1996, Cancer Immunol Immunother 42:71-80; Barratt-Boyes et al, 1996, Cancer Immunol Immunother 43:142-151). One of the most striking features of the MUC-1 molecule is its extensive O-linked glycosylation. There are five O-linked glycosylation sites available within each MUC-1 VNTR monomer.

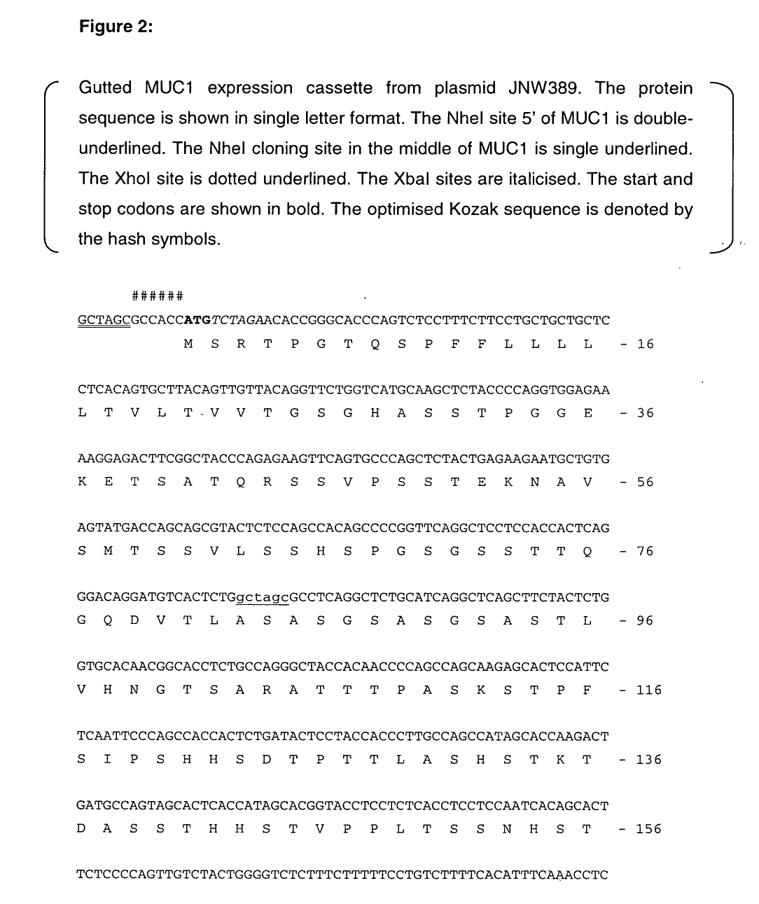

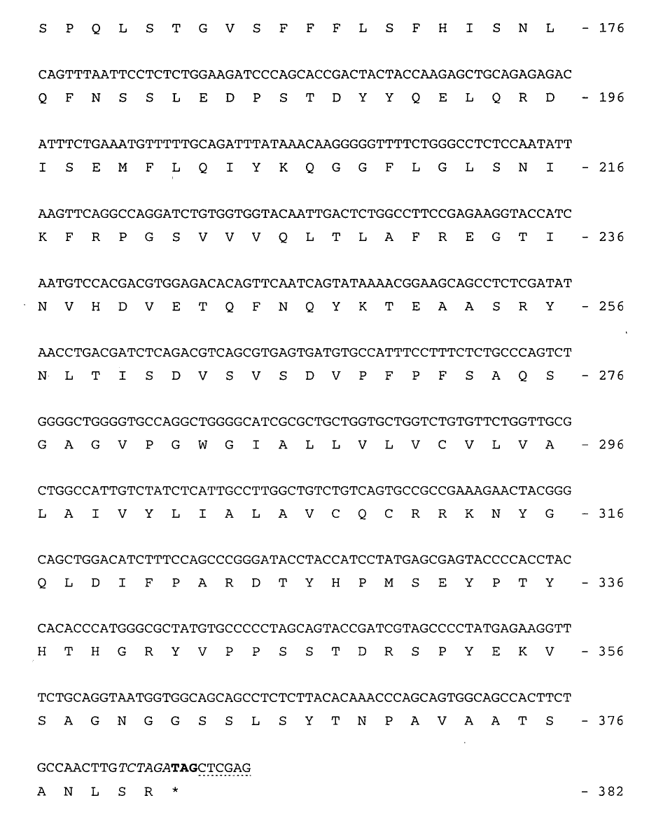

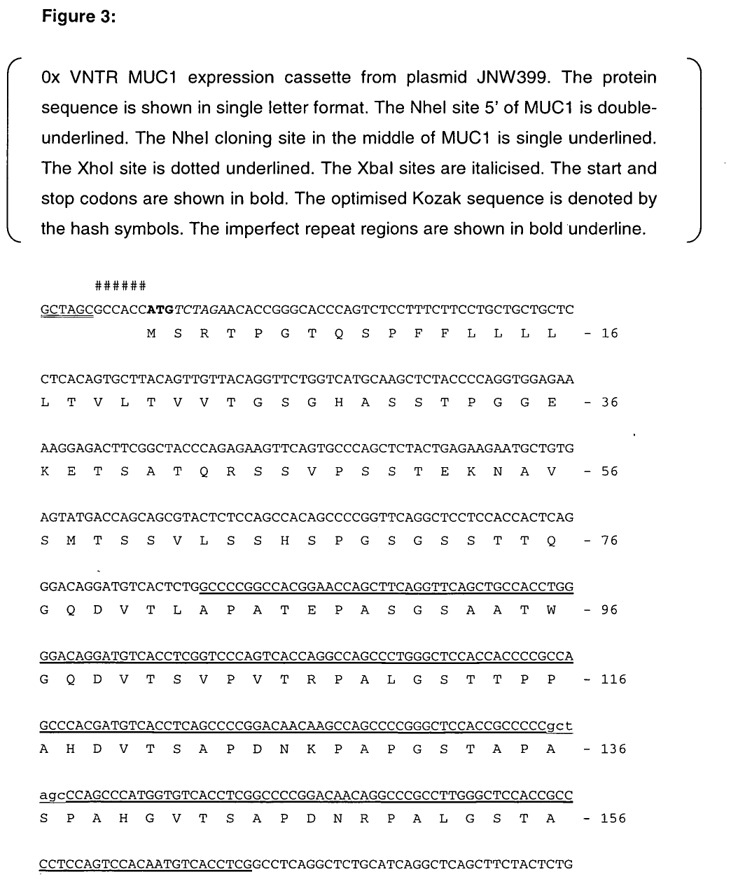

The VNTR can be characterised as typical or perfect repeats having a sequence: and imperfect (atypical) repeats which has minor variation for the perfect repeat comprising two to three differences over the 20 amino acids. The imperfect repeat in wild type-Muc-1 flank the perfect repeat region. In malignant carcinomas arising by neoplastic transformation of these epithelial cells, several changes affect the expression of MUC-1. The polarised expression of the protein is lost, and it is found spread over the whole surface of the transformed cell. The total amount of MUC-1 is also increased, often by 10-fold or more (Strous & Dekker, 1992, Crit Rev Biochem Mol Biol 27:57-92). Most significantly, the quantity and quality of the O-linked carbohydrate chains changes markedly. Fewer serine and threonine residues are glycosylated. Those carbohydrate chains that are found are abnormally shortened, creating the tumour-associated carbohydrate antigen STn (Lloyd et al, 1996, J Biol Chem, 271:33325-33334). As a result of these glycosylation changes, various epitopes on the peptide chain of MUC-1 which were previously screened by the carbohydrate chains become accessible. One epitope which becomes accessible in this way is formed by the sequence APDTR (Ala 8-Arg 12 in FIG. 2) present in each 20 amino acid VNTR perfect monomer (Burchell et al, 1989, Int J Cancer 44:691-696).

It is apparent that these changes in MUC-1 mean that a vaccine that can activate the immune system against the form of MUC-1 expressed on tumours may be effective against epithelial cell tumours, and indeed other cell types where MUC-1 is found, such as T cell lymphocytes. One of the main effector mechanisms used by the immune system to kill cells expressing abnormal proteins is a cytotoxic T lymphocyte immune response (CTL's) and this response is desirable in a vaccine to treat tumours, as well as an antibody response. A good vaccine will activate all arms of the immune response. However, current carbohydrate and peptide vaccines such as Theratope or BLP25 (Biomira Inc, Edmonton, Canada) preferentially activate one arm of the immune response—a humoral and cellular response respectively, and better vaccine designs are desirable to generate a more balanced response.

Nucleic acid vaccines provide a number of advantages over conventional protein vaccination, in that they are easy to produce in large quantity. Even at small doses they have been reported to induce strong immune responses, and can induce a cytotoxic T lymphocyte immune response as well as an antibody response.

The full-length MUC-1, however, is very difficult to work with due to the highly repetitive sequence, since it is highly susceptible to recombination, such recombination events cause significant development difficulties. Additionally the GC rich nature of the VNTR region makes sequencing difficult. Further for regulatory reasons—it is necessary to fully characterise the DNA construct. It is highly problematic to sequence a molecule with such a high frequency repeating structure. Given that it is unknown precisely how many repeat units are in wild type MUC-1 this inability to precisely characterise full-length MUC-1 makes this unacceptable for regulatory approval.

MUC-1 VNTR regions are thought to contain immunodominant epitopes. Surprisingly the present inventors have found that it is possible to produce immunogenic MUC-1 constructs capable of raising an immune response that is capable of recognising MUC-1 expressing tumours said constructs being devoid of any perfect VNTR units.

### SUMMARY OF THE INVENTION

The present invention provides a nucleic acid sequence encoding a MUC-1 derivative which is capable of raising an immune response in vivo, said immune response being capable to recognising a MUC-1 expressing tumour, wherein the encoded MUC-1 derivative is devoid of any perfect VNTR units.

Such constructs, are surprisingly, capable of raising both a cellular and also an antibody response that recognise MUC-1 expressing tumour cells.

In an embodiment of the invention, the derivative is also devoid of the perfect repeat.

The constructs can also be used as an intermediate to insert foreign epitopes and altered VNTR units, such as reduced glycosylation-mutants. Typical foreign epitopes that may be incorporated include T-helper epitopes such as derived from bacterial proteins and toxins and from viral sources, eg. T-Helper epitopes from Diptheria or Tetanus, eg P2 and P30 or epitopes from Hep B case antigen.

In yet further embodiments, the invention contemplates nucleic acids that encode for fusion proteins that have heterologous protein at the N or C terminus of the MUC-1 constructs of the invention. Such fusion partners, provide T-helper epitopes or are capable of eliciting a re-call response.

Examples of these include Tetanus, Diptheria, Tuberculosis or hepatitis proteins, such as Tetanus or Diptheria toxin, in particular a fragment of Tetanus toxin that incorporates the P2 and/or P30 epitope. An example of a Mycobacterium tuberculosis peptide is Ra12 corresponding to amino-acids 192 to 323 of Mtb32a (Skeiky et al Infection and Immunity (1999) 67: 3998-4007). Hepatitis B core antigen is illustrative of yet another embodiment.

Other preferred immunological fusion partners include protein D, typically the N terminal 1/3 (eg N terminal 1-109); LYTA or portion thereof (preferably the C-terminal portion) from Streptococcus pneumoniae (Biotechnology) 10: 795-798, 1992).

Such constructs are useful in vaccines which direct the immune response away from the VNTR region. Thus they can be used with VNTR based vaccines, for example, BLP-25 from Biomira, (current opinion in Mol. Ther 2001 3 (1) p 102-105) to elicit a balanced immune response to both VNTR and non-VNTR regions.

In further aspect of the invention the nucleic acid sequence is a DNA sequence in the form of a plasmid. Preferably the plasmid is super-coiled. Proteins encoded by such nucleotide sequences are novel and form an aspect of the invention.

In a further aspect of the invention there is provided a pharmaceutical composition comprising a nucleic acid sequence or protein as herein described and a pharmaceutical acceptable excipient, diluent or carrier.

Preferably the carrier is a gold bead and the pharmaceutical composition is amenable to delivery by particle mediated drug delivery.

In yet a further embodiment, the invention provides the pharmaceutical composition and nucleic acid constructs for use in medicine. In particular, there is provided a nucleic acid construct of the invention, in the manufacture of a medicament for use in the treatment or prophylaxis of MUC-1 expressing tumours.

The invention further provides for methods of treating a patient suffering from or susceptible to MUC-1 expressing tumour, particularly carcinoma of the breast, lung (particularly non-small cell lung carcinoma), gastric and other GI (gastrointestinal) carcinomas by the administration of a safe and effective amount of a composition or nucleic acid as herein described.

In yet a further embodiment the invention provides a method of producing a pharmaceutical composition as herein described by admixing a nucleic acid construct or protein of the invention with a pharmaceutically acceptable excipient, diluent or carrier.

### DETAILED DESCRIPTION OF THE INVENTION

As described herein the nucleic acid constructs of the invention, have no perfect repeats. The wild type MUC-1 molecule contains a signal sequence, a leader sequence, imperfect or atypical VNTR, the perfect VNTR region, a further atypical VNTR, a non-VNTR extracellular domain a transmembrane domain and a cytoplasmic domain.

Preferred embodiments of the invention have no imperfect repeats, or one imperfect repeat, more preferably, two, three or four imperfect repeats, but no perfect repeat units.

The non-VNTR extracellular domain is approximately 80 amino acids, 5′ of VNTR and 190-200 amino acids 3′ VNTR. All constructs of the invention comprise at least one epitope from this region. An epitope is typically formed from at least seven amino acid sequence. Accordingly the constructs of the present invention include at least one epitope from the non VNTR extra-cellular domain. Preferably substantially all or more preferably all of the non-VNTR domain is included. It is particularly preferred that construct contains the epitope comprised by the sequence FLSFHISNL; NSSLEDPSTDYYQELQRDISE, or NLTISDVSV. More preferred is that two, preferable all three, epitope sequences are incorporated in the construct.

In a preferred embodiment the constructs comprise an N-terminal leader sequence. The signal sequence, transmembrane domain and cytoplasmic domain are individually all optionally present or deleted.

Preferred constructs according to the invention are:
1) Truncated MUC-1 (ie Full MUC-1 with no perfect repeats)
2) Truncated MUC-1 Δss (As I, but also devoid of signal sequence)
3) Truncated MUC-1 ΔTM ΔCYT (As 1, but devoid of Transmembrane and cytoplasmic domains)
4) Truncated MUC-1 Δss ΔTM ΔCYT (As 3, but also devoid of signal sequence)

Also preferred are equivalent constructs of 1 to 4 above, but devoid of imperfect MUC-1 repeat units. Such constructs are referred to as gutted-MUC-1. In an embodiment one or more of the imperfect VNTR units is mutated to reduce the potential for glycosylation, by altering a glycosylation site. The mutation is preferably a replacement, but can be an insertion or a deletion. Typically at least one threonine or seriene is substituted with valine, Isoleucine, alanine, asparagine, phenylalanine or tryptophan. It is thus preferred that at least one, preferably 2 or 3 or more are substituted with an amino acid as noted above.

In a further embodiment, the gutted MUC-1 nucleic acid is provided with a restriction site at the junction of the leader sequence and the extracellular domain. Typically this restriction site is a Nhe1 site. This can be utilised as a cloning site to insert sequences encoding for other peptides including, for example glycosylation mutants (ie. VNTR regions mutated to remove O-glycosylation sites), or heterologous sequences that encode T-Helper epitopes such as P2 or P30.

According to a further aspect of the invention, an expression vector is provided which comprises and is capable of directing the expression of a polynucleotide sequence according to the invention. The vector may be suitable for driving expression of heterologous DNA in bacterial insect or mammalian cells, particularly human cells.

According to a further aspect of the invention, a host cell comprising a polynucleotide sequence according to the invention, or an expression vector according the invention is provided. The host cell may be bacterial, e.g. E. coli, mammalian, e.g. human, or may be an insect cell. Mammalian cells comprising a vector according to the present invention may be cultured cells transfected in vitro or may be transfected in vivo by administration of the vector to the mammal.

The present invention further provides a pharmaceutical composition comprising a polynucleotide sequence according to the invention. Preferably the composition comprises a DNA vector. In preferred embodiments the composition comprises a plurality of particles, preferably gold particles, coated with DNA comprising a vector encoding a polynucleotide sequence of the invention which the sequence encodes a MUC-1 amino acid sequence as herein described. In alternative embodiments, the composition comprises a pharmaceutically acceptable excipient and a DNA vector according to the present invention.

The composition may also include an adjuvant, or be administered either concomitantly with or sequentially with an adjuvant or immuno-stimulatory agent.

Thus it is an embodiment of the invention that the vectors of the invention be utilised with immunostimulatory agent. Preferably the immunostimulatory agent is administered at the same time as the nucleic acid vector of the invention and in preferred embodiments are formulated together. Such immunostimulatory agents include, (but this list is by no means exhaustive and does not preclude other agents): synthetic imidazoquinolines such as imiquimod [S-26308, R-837], (Harrison, et al. ‘Reduction of recurrent HSV disease using imiquimod alone or combined with a glycoprotein vaccine’, Vaccine 19: 1820-1826, (2001)); and resiquimod [S-28463, R-848] (Vasilakos, et al. ‘Adjuvant activites of immune response modifier R-848: Comparison with CpG ODN’, Cellular immunology 204: 64-74 (2000).), Schiff bases of carbonyls and amines that are constitutively expressed on antigen presenting cell and T-cell surfaces, such as tucaresol (Rhodes, J. et al. ‘Therapeutic potentiation of the immune system by costimulatory Schiff-base-forming drugs’, Nature 377: 71-75 (1995)), cytokine, chemokine and co-stimulatory molecules as either protein or peptide, this would include pro-inflammatory cytokines such as Interferon, particular Interferon alpha, GM-CSF, IL-1 alpha, IL-1 beta, TGF-alpha and TGF-beta, Th1 inducers such as interferon gamma, IL-2, IL-12, IL-15, IL-18 and IL-21, Th2 inducers such as IL-4, IL-5, IL-6, IL-10 and IL-13 and other chemokine and co-stimulatory genes such as MCP-1, MIP-1 alpha, MIP-1 beta, RANTES, TCA-3, CD80, CD86 and CD40L, other immunostimulatory targeting ligands such as CTLA-4 and L-selectin, apoptosis stimulating proteins and peptides such as Fas, (49), synthetic lipid based adjuvants, such as vaxfectin, (Reyes et al., ‘Vaxfectin enhances antigen specific antibody titres and maintains Th1 type immune responses to plasmid DNA immunization’, Vaccine 19: 3778-3786) squalene, alpha-tocopherol, polysorbate 80, DOPC and cholesterol, endotoxin, [LPS], Beutler, B., ‘Endotoxin, ‘Toll-like receptor 4, and the afferent limb of innate immunity’, Current Opinion in Microbiology 3: 23-30 (2000)); CpG oligo- and di-nucleotides, Sato, Y. et al., ‘Immunostimulatory DNA sequences necessary for effective intradermal gene immunization’, Science 273 (5273): 352-354 (1996). Hemmi, H. et al., ‘A Toll-like receptor recognizes bacterial DNA’, Nature 408: 740-745, (2000) and other potential ligands that trigger Toll receptors to produce Th1-inducing cytokines, such as synthetic Mycobacterial lipoproteins, Mycobacterial protein p19, peptidoglycan, teichoic acid and lipid A. Other bacterial immunostimulatory proteins such as Cholera Toxin, E. coli Toxin and mutant toxoids thereof can be utilised.

Certain preferred adjuvants for eliciting a predominantly Th1-type response include, for example, a Lipid A derivative such as monophosphoryl lipid A, or preferably 3-de-O-acylated monophosphoryl lipid A. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins.

Also provided are the use of a polynucleotide or protein according to the invention, or of a vector according to the invention, in the treatment or prophylaxis of MUC-1 expressing tumour or metastases.

The present invention also provides methods of treating or preventing MUC-1 expressing tumour, any symptoms or diseases associated therewith including metastases, comprising administering an effective amount of a polynucleotide, a vector or a pharmaceutical composition according to the invention. Administration of a pharmaceutical composition may take the form of one or more individual doses, for example in a “prime-boost” therapeutic vaccination regime. In certain cases the “prime” vaccination may be via particle mediated DNA delivery of a polynucleotide according to the present invention, preferably incorporated into a plasmid-derived vector and the “boost” by administration of a recombinant viral vector comprising the same polynucleotide sequence, or boosting with the protein in adjuvant. Conversly the priming may be with the viral vector or with a protein formulation typically a protein formulated in adjuvant and the boost a DNA vaccine of the present invention.

As discussed above, the present invention includes expression vectors that comprise the nucleotide sequences of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. Molecular Cloning: a Laboratory Manual. 2^{nd} Edition. CSH Laboratory Press. (1989).

Preferably, a polynucleotide of the invention, or for use in the invention in a vector, is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be, for example, plasmids, artificial chromosomes (e.g. BAC, PAC, YAC), virus or phage vectors provided with an origin of replication, optionally a promoter for the expression of the polynucleotide and optionally a regulator of the promoter. The vectors may contain one or more selectable marker genes, for example an ampicillin or kanamycin resistance gene in the case of a bacterial plasmid or a resistance gene for a fungal vector. Vectors may be used in vitro, for example for the production of DNA or RNA or used to transfect or transform a host cell, for example, a mammalian host cell e.g. for the production of protein encoded by the vector. The vectors may also be adapted to be used in vivo, for example in a method of DNA vaccination or of gene therapy.

Promoters and other expression regulation signals may be selected to be compatible with the host cell for which expression is designed. For example, mammalian promoters include the metallothionein promoter, which can be induced in response to heavy metals such as cadmium, and the β-actin promoter. Viral promoters such as the SV40 large T antigen promoter, human cytomegalovirus (CMV) immediate early (IE) promoter, rous sarcoma virus LTR promoter, adenovirus promoter, or a HPV promoter, particularly the HPV upstream regulatory region (URR) may also be used. All these promoters are well described and readily available in the art.

A preferred promoter element is the CMV immediate early promoter devoid of intron A, but including exon 1. Accordingly there is provided a vector comprising a polynucleotide of the invention under the control of HCMV IE early promoter.

Examples of suitable viral vectors include herpes simplex viral vectors, vaccinia or alpha-virus vectors and retroviruses, including lentiviruses, adenoviruses and adeno-associated viruses. Gene transfer techniques using these viruses are known to those skilled in the art. Retrovirus vectors for example may be used to stably integrate the polynucleotide of the invention into the host genome, although such recombination is not preferred. Replication-defective adenovirus vectors by contrast remain episomal and therefore allow transient expression. Vectors capable of driving expression in insect cells (for example baculovirus vectors), in human cells or in bacteria may be employed in order to produce quantities of the HIV protein encoded by the polynucleotides of the present invention, for example for use as subunit vaccines or in immunoassays. The polynucleotides of the invention have particular utility in viral vaccines as previous attempts to generate full-length vaccinia constructs have been unsuccessful.

The polynucleotides according to the invention have utility in the production by expression of the encoded proteins, which expression may take place in vitro, in vivo or ex vivo. The nucleotides may therefore be involved in recombinant protein synthesis, for example to increase yields, or indeed may find use as therapeutic agents in their own right, utilised in DNA vaccination techniques. Where the polynucleotides of the present invention are used in the production of the encoded proteins in vitro or ex vivo, cells, for example in cell culture, will be modified to include the polynucleotide to be expressed. Such cells include transient, or preferably stable mammalian cell lines. Particular examples of cells which may be modified by insertion of vectors encoding for a polypeptide according to the invention include mammalian HEK293T, CHO, HeLa, 293 and COS cells. Preferably the cell line selected will be one which is not only stable, but also allows for mature glycosylation and cell surface expression of a polypeptide. Expression may be achieved in transformed oocytes. A polypeptide may be expressed from a polynucleotide of the present invention, in cells of a transgenic non-human animal, preferably a mouse. A transgenic non-human animal expressing a polypeptide from a polynucleotide of the invention is included within the scope of the invention.

The invention further provides a method of vaccinating a mammalian subject which comprises administering thereto an effective amount of such a vaccine or vaccine composition. Most preferably, expression vectors for use in DNA vaccines, vaccine compositions and immunotherapeutics will be plasmid vectors.

DNA vaccines may be administered in the form of “naked DNA”, for example in a liquid formulation administered using a syringe or high pressure jet, or DNA formulated with liposomes or an irritant transfection enhancer, or by particle mediated DNA delivery (PMDD). All of these delivery systems are well known in the art. The vector may be introduced to a mammal for example by means of a viral vector delivery system.

The compositions of the present invention can be delivered by a number of routes such as intramuscularly, subcutaneously, intraperitonally or intravenously.

In a preferred embodiment, the composition is delivered intradermally. In particular, the composition is delivered by means of a gene gun (particularly particle bombardment) administration techniques which involve coating the vector on to a bead (eg gold) which are then administered under high pressure into the epidermis; such as, for example, as described in Haynes et al, J Biotechnology 44: 37-42 (1996).

In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide, are accelerated to high speed within a helium gas jet generated by a hand held device, propelling the particles into a target tissue of interest, typically the skin. The particles are preferably gold beads of a 0.4-4.0 μm, more preferably 0.6-2.0 μm diameter and the DNA conjugate coated onto these and then encased in a cartridge or cassette for placing into the “gene gun”.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

The vectors which comprise the nucleotide sequences encoding antigenic peptides are administered in such amount as will be prophylactically or therapeutically effective. The quantity to be administered, is generally in the range of one picogram to 1 milligram, preferably 1 picogram to 10 micrograms for particle-mediated delivery, and 10 micrograms to 1 milligram for other routes of nucleotide per dose. The exact quantity may vary considerably depending on the weight of the patient being immunised and the route of administration.

It is possible for the immunogen component comprising the nucleotide sequence encoding the antigenic peptide, to be administered on a once off basis or to be administered repeatedly, for example, between 1 and 7 times, preferably between 1 and 4 times, at intervals between about 1 day and about 18 months. Once again, however, this treatment regime will be significantly varied depending upon the size of the patient, the disease which is being treated/protected against, the amount of nucleotide sequence administered, the route of administration, and other factors which would be apparent to a skilled medical practitioner. The patient may receive one or more other anti cancer drugs as part of their overall treatment regime.

Suitable techniques for introducing the naked polynucleotide or vector into a patient also include topical application with an appropriate vehicle. The nucleic acid may be administered topically to the skin, or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by use of facilitating agents such as bupivacaine, either separately or included in the DNA formulation. Other methods of administering the nucleic acid directly to a recipient include ultrasound, electrical stimulation, electroporation and microseeding which is described in U.S. Pat. No. 5,697,901.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered.

A nucleic acid sequence of the present invention may also be administered by means of transformed cells. Such cells include cells harvested from a subject. The naked polynucleotide or vector of the present invention can be introduced into such cells in vitro and the transformed cells can later be returned to the subject. The polynucleotide of the invention may integrate into nucleic acid already present in a cell by homologous recombination events. A transformed cell may, if desired, be grown up in vitro and one or more of the resultant cells may be used in the present invention. Cells can be provided at an appropriate site in a patient by known surgical or microsurgical techniques (e.g. grafting, micro-injection, etc.)

### EXAMPLES

1. Construction of Gutted MUC-1 and 0x VNTR MUC-1 Vectors

The starting point for the construction of the gutted (i.e. No imperfect or perfect repeats) and 0x VNTR MUC-1, no perfect repeats vectors was the plasmid pcDNA3-FL-MUC1 (ICRF, London). The MUC-1 fragment was excised as a BamHI fragment and cloned into pcDNA3.1(+)/Hygro generating vector JNW278. The VNTR units were removed by FseI digestion and the vector re-ligated to give JNW283. This plasmid contains MUC-1 with a single VNTR unit. The 3′ section of MUC-1 was PCR amplified using primers and 2062MUC1 and ATR5′FOR for gutted MUC-1 and 2062MUC1 and atypical TRs FOR for 0x VNTR MUC-1 (primer sequences are listed in Appendix A). The PCR products were purified and digested with NheI-XhoI and cloned into the NheI-XhoI site of pVAC restricted with NheI-XhoI. This generated the following intermediate vectors JNW348 and JNW353.

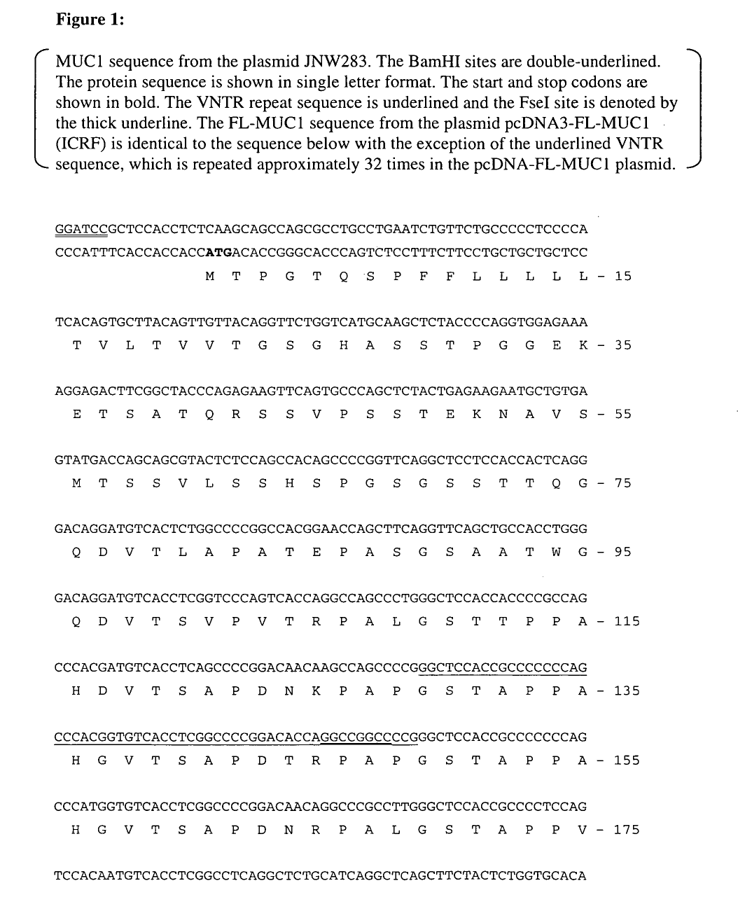

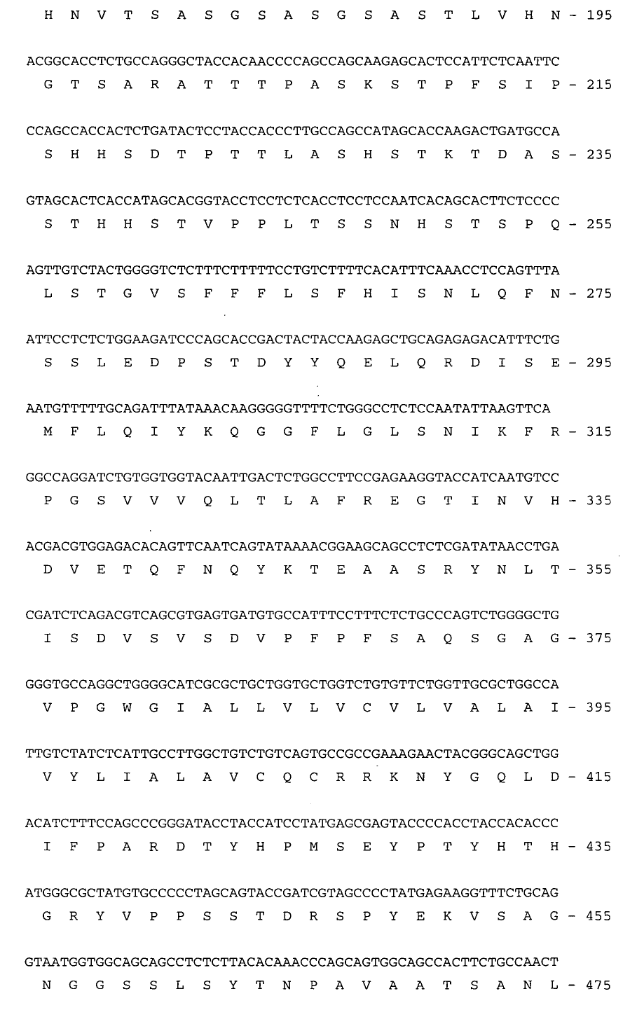

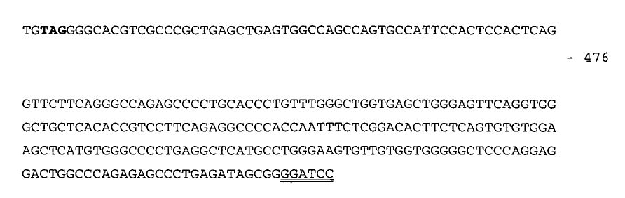

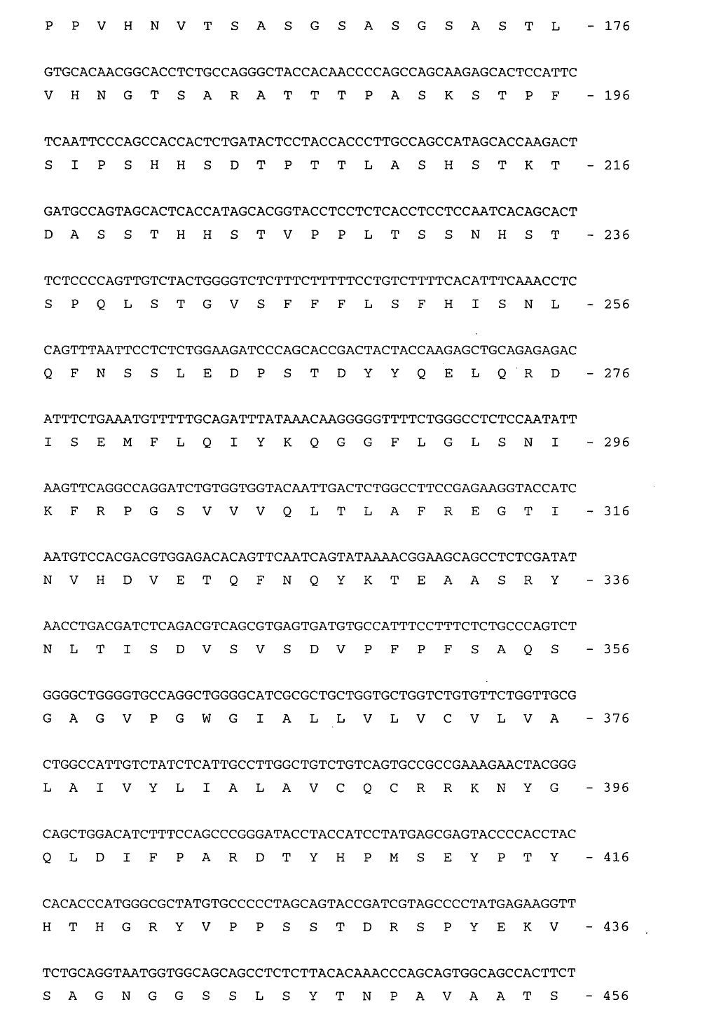

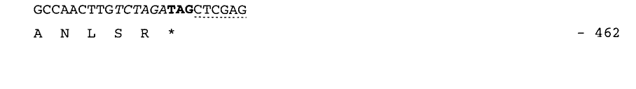

The 5′ section of MUC-1 was PCR amplified using primers 2060MUC1 and ΔTR3′REV for gutted MUC-1 and 2060MUC1 and atypical TRs REV for 0x VNTR MUC-1. The resulting PCR fragments were cloned into vectors JNW348 and JNW353 respectively, digested with NheI and dephosphorylated with calf intestinal phosphatase. This cloning procedure generated vectors JNW389 (gutted MUC-1) and JNW399 (0x VNTR MUC-1). The sequences of the MUC-1 cassette in JNW283, JNW389 and JNW399 are shown in FIGS. 1-3 and were confirmed by sequencing using primers 2004MUC1-2014MUC1 (see Appendix A). Gutted MUC-1 contains no perfect or imperfect repeats. 0x VNTR MUC-1 contains only imperfect repeats (no perfect repeats are present).

Note: perfect repeats are based on the sequence shown in Engelmann et al. (2001) JBC 276(30) 27764-27769 and are as follows. The 20 amino acid VNTR unit is shown. Amino acid substitutions are only allowed at the underlined residues.

```
                          T
                          A
      E S  Q
A P D T R P A P G S  T  A  P  P A  H  G  V  T  S
1 2 3 4 5 6 7 8 9 10 11 12 13 14 15 16 17 18 19 20
```

Imperfect repeats have different amino acid substitutions to the consensus sequence above with 55-90% identity at the amino acid level. The four imperfect repeats are shown below, with the substitutions underlined:

APDTRPAPGSTAPPAHGVTS - perfect repeat
APATEPASGSAATWGQDVTS - imperfect repeat 1
VPVTRPALGSTTPPAHDVTS - imperfect repeat 2
APDNKPAPGSTAPPAHGVTS - imperfect repeat 3
APDNRPALGSTAPPVHNVTS - imperfect repeat 4

2. Comparison of Antibody Responses

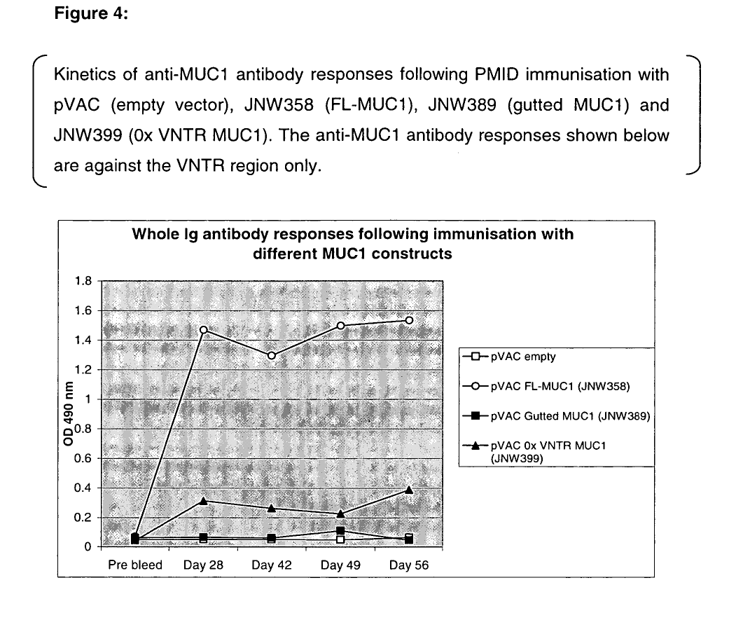

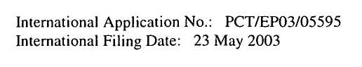

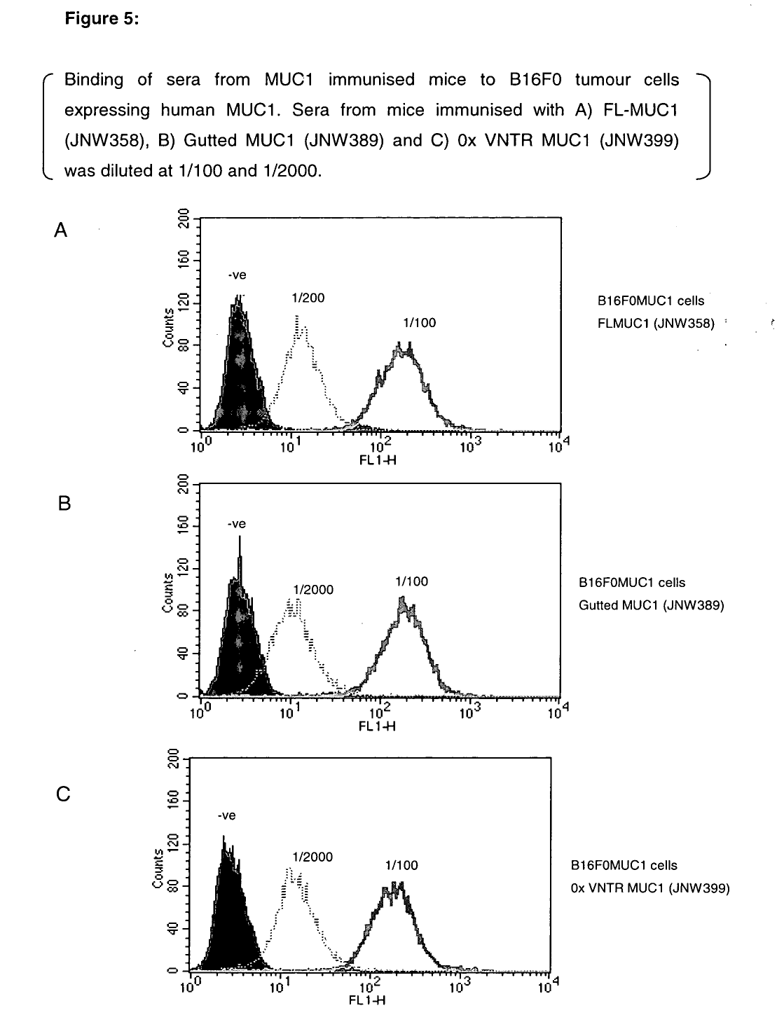

The anti-MUC-1 antibody responses to the immunodominant epitope following PMID immunisation with pVAC (empty vector), JNW358 (FL-MUC1), JNW 389 (Gutted MUC-1) and JNW399 (0x VNTR MUC-1) are shown in FIG. 4. The results show that as predicted Gutted MUC-1 does not induce an antibody response to the immunodominant MUC-1 epitope. In contrast, 0x VNTR MUC-1 (JNW399) induces a weak antibody response suggesting a degree of cross-reactivity between the imperfect and perfect repeat sequences at the antibody level.

To investigate the antibody responses to the remainder of the extracellular domain, sera from immunised mice was tested by flow cytometry. The target cells were B16F0MUC1, a tumour cell line expressing human MUC-1. The results show that JNW389 and JNW399 induce a strong antibody response which is capable of recognising the human form of MUC-1. Titration of these sera (1/100 and 1/2000) indicates that the antibody response induced by immunisation with JNW389 and JNW399 is comparable to that of FL-MUC1. This data suggests that the antibody response to non-VNTR extracellular domain may be an important feature of a MUC-1 immunotherapeutic vaccine.

3. Cellular Responses to FL-MUC1, 0x VNTR MUC-1 and Gutted MUC-1

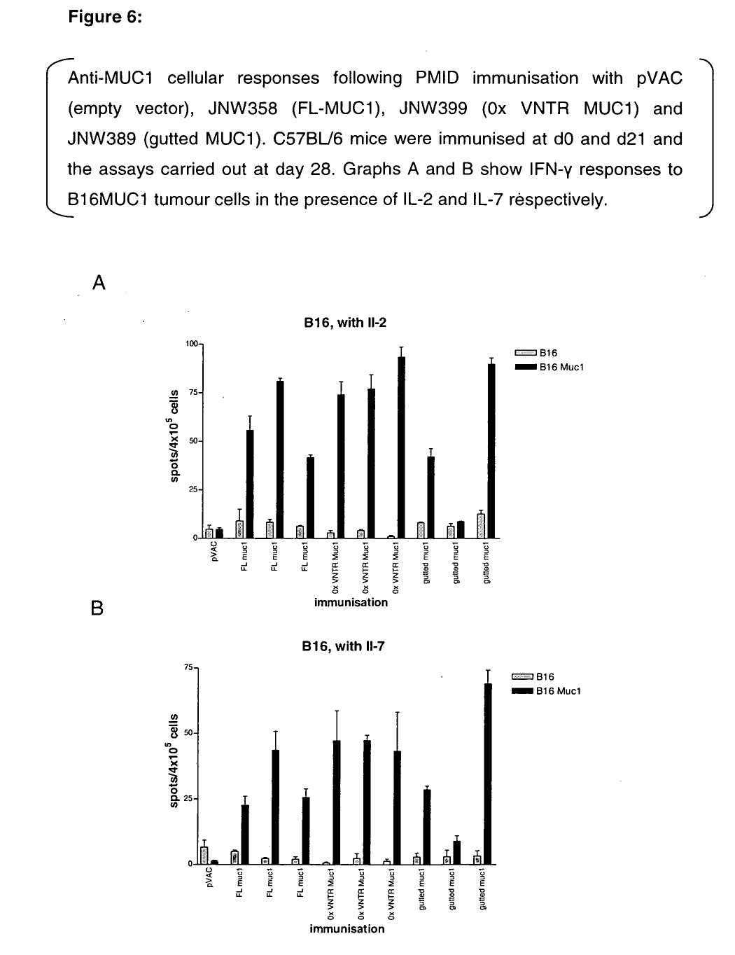

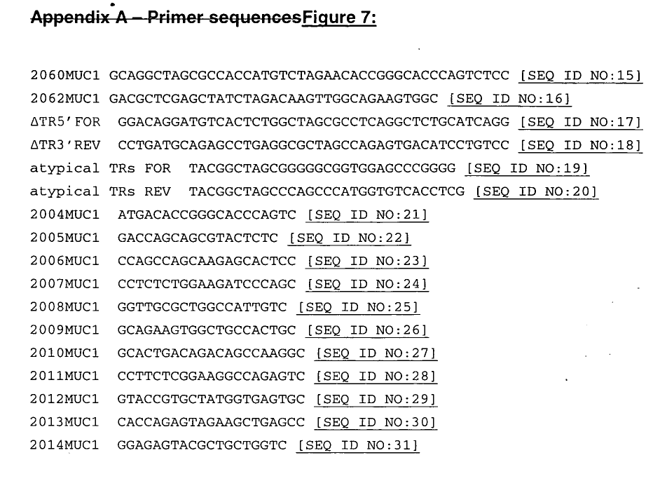

The cellular responses following immunisation with pVAC (empty vector), JNW358 (FL-MUC1), JNW399 (0x VNTR MUC-1) and JNW389 (gutted MUC-1) were assessed by ELISPOT following a primary immunisation by PMID at day 0 and a boost at day 21. Assays were carried out 7 days post boost. FIG. 6 shows that the cellular responses to B16 MUC1 tumour cells are equivalent following vaccination with either FL-MUC1, 0x VNTR or gutted MUC-1, suggesting that the absence of the VNTR region is not detrimental to the induction of a robust cellular response against tumour cells expressing human MUC-1. In conjunction with the antibody results, these data suggest that the non-VNTR regions of MUC-1 should be an integral component in the design of a MUC-1 vaccine.

4. Construction of Gutted and Ox VNTR Constructs Containing Glycosylation Mutants of MUC-1

The starting point for the construction of these vectors is the plasmids JNW389 (Gutted MUC-1) and JNW399 (0x VNTR MUC-1). These plasmids can be linearised with NheI and dephosphorylated with calf intestinal phosphatase.

The glycosylation mutant VNTR fragments (2TRs—two tandem repeats) can be isolated by NheI digestion of the following plasmids (See WO/01/46228).

| | |
|---|---|
| 2TR-WT | pVAC1.ss2.WT.MUC1.HepB |
| MUT4 | pVAC1.ss2.MUT4.MUC1.HepB |
| MUT5N | pVAC1.ss2.MUT5N.MUC1.HepB |

These fragments can be ligated into JNW389/JNW399 linearised as described above. Clones containing the fragment in the correct orientation can be identified by sequencing using the following sequencing primers (2004MUC1, 2005MUC1, 2013MUC1, 2012MUC1) which flank the NheI cloning site of JNW389 and JNW399.

Other glycosylation mutants of MUC-1 (Mut5I, V, W, P, A) can be inserted into the linearised 3NW389 and JNW399 vectors using oligos which encode the mutated VNTR sequence. The oligos are phosphorylated and annealed as follows:

| | |
|---|---|
| 10 pmol | Primer A |
| 10 pmol | Primer B |
| 1× | T4 DNA ligase buffer |
| 10 U | T4 Polynucleotide kinase |
| to 20 μl Water | |

2 hrs, 37° C.
95° C. for 2 mins
Anneal oligos by decreasing temperature 0.1° C./s
Hold at 4° C. Freeze until required.

The annealed oligos are ligated with the NheI linearised JNW389 and JNW399. Clones can be identified by sequencing with 2004MUC1, 2005MUC1, 2012MUC1 and 2013MUC1.

Mix and Match

Since all the above glycosylation mutants VNTR sequences are encoded on an NheI cassette, and the vectors JNW389 and JNW399 have an appropriately positioned NheI site, it is possible to build up MUC-1 molecules containing various different glycosylation mutants and different numbers of VNTR units (1-10), to achieve the desired antibody response.

5. Expression of the Gutted/0x VNTR Constructs Containing Glycosylation Mutants

Constructs can be transiently transfected into CHO cells. For the gutted MUC-1 construct, the presence of the glycosylation VNTR sequences can be confirmed using the antibody ATR1 (either by FACS or Western blot).

Confirmation That the Gutted/0x VNTR Constructs Containing Glycosylation Mutants Induce an Antibody Response Which Will Recognise Tumour Cells

Constructs can be used to immunise mice by PMID at days 0, 21 and 42. Sera will be taken at approx. day 49 and used in an ELISA. Similarly, using a FACS based assay and tumour cells expressing human MUC-1 (e.g. B16F0MUC1, T47D, RMA-MUC1, EL4-MUC1), sera from immunised mice will be tested for its ability to recognise the tumour form of human MUC-1.

1. A nucleic acid molecule encoding a MUC-1 derivative that is devoid of all perfect repeats.

2. A nucleic acid molecule encoding a MUC-1 derivative as claimed in claim 1 further devoid of the imperfect repeats.

3. A nucleic acid molecule as claimed in claim 1 further devoid of the signal sequence.

4. A nucleic acid molecule as claimed in claim 1 further devoid of a functional transmembrane domain, and/or cytoplasmic domain.

5. A nucleic acid molecule as claimed in claim 1 that encodes one or more of the sequence from the group: FLSFHISNL[SEQ ID NO:1]; NSSLEDPSTDYYQELQRDISE[SEQ ID NO:2]; and NLTISDVSV[SEQ ID NO:3].

6. A nucleic acid molecule as claimed in claim 1 additionally comprising a heterologous sequence that encodes a T-Helper epitope.

7. A nucleic acid molecule as claimed in claim 1 that is a DNA molecule.

8. A plasmid comprising the DNA molecule of claim 7.

9. A protein encoded by a nucleic acid molecule as claimed in claim 1.

10. A pharmaceutical composition comprising a nucleic acid as claimed in claim 1 and a pharmaceutical acceptable excipient, diluent or carrier.

11. A pharmaceutical composition as claimed in claim 10 wherein the carrier is microparticle.

12. A pharmaceutical composition as claimed in claim 11 wherein the microparticle is gold.

13. A pharmaceutical composition as claimed in claim 10 comprising an adjuvant.

14. A nucleic acid as claimed in any claim 1 for use in medicine.

15. Use of a nucleic acid as claimed in claim 1 in the preparation of a medicament for the treatment or prevention MUC-1 expressing tumours.

16. Use of a protein as claimed in claim 8 in the manufacture of a medicament for the treatment or prevention of MUC-1 expressing tumours.

17. A method of treating or preventing tumours, comprising administering a safe and effective amount of a nucleic acid as claimed in claim 1.
